(19) United States
(12) Patent Application Publication
(10) Pub. No.: US 2003/0236512 A1
(43) Pub. Date: Dec. 25, 2003

(54) Absorbent core with folding zones for absorbency distribution

(76) Inventor: Andrew A. Baker, Lawrenceville, GA (US)

Correspondence Address: Christopher C. Campbell, Esq., Hunton & Williams, Suite 1200, 1900 K Street, NW, Washington, DC, 20006-1109, US

(21) Appl. No.: 10/173,630

(22) Filed: Jun. 19, 2002

(51) Int. Cl. A61F 13/15; A61F 13/20

(52) U.S. Cl. 604/385.01

(57) ABSTRACT

The features of the invention generally may be achieved by an absorbent core having a longitudinal dimension and a lateral dimension, and a fibrous matrix with superabsorbent particles distributed within the fibrous matrix. The absorbent core has a folded zone that has at least one laterally-extending fold. The folded zone extends through at least a portion of the lateral dimension of the absorbent core and through at least a portion of the longitudinal dimension of the absorbent core. An apparatus for manufacturing such an absorbent core and a garment incorporating such an absorbent core also are provided.

### FIELD OF THE INVENTION

The present invention generally relates to absorbent garments and their manufacture. In particular, this invention relates to an absorbent core having folded high absorbency zones and a method for manufacturing such an absorbent core.

### BACKGROUND OF THE INVENTION

Traditionally, disposable absorbent garments, such as infant diapers, training pants, adult incontinence briefs and other such products were constructed with a moisture-impervious outer or backing sheet (often referred to as a “backsheet”), a moisture-pervious body-contacting inner liner sheet (often referred to as a “topsheet”), and a moisture-absorbent core sandwiched and encased between the topsheet and backsheet.

The moisture-absorbent cores of absorbent garments typically use a fibrous matrix of material into which particles of superabsorbent material, in the form of granules, beads, fibers, flakes and so on, are dispersed. Such superabsorbent materials generally are polymeric gelling materials that are capable of absorbing large quantities of liquids such as water and body wastes relative to their weight, and can retain such absorbed materials even under moderate pressure. The use of such superabsorbents, however, has given rise to problems with the design of absorbent garments. One problem with superabsorbents it that they may impede the rapid absorption of fluids by a phenomenon known as “gel blocking.” Another problem with superabsorbents is that they are relatively expensive.

The ability of a superabsorbent material to absorb liquid depends, at least in part, upon the form, position, and/or manner in which the superabsorbent particles are incorporated into the absorbent core. Whenever a particle of superabsorbent material in an absorbent core is wetted, it swells and forms a gel. Gel formation can block liquid transmission into the interior of the absorbent core, a phenomenon called “gel blocking.” Gel blocking in portions of the absorbent core that typically receive the initial liquid contact (“insult points”) may prevent liquid from rapidly diffusing or wicking past the “blocking” superabsorbent particles and into the rest of the absorbent core; further imbibition of liquid by the absorbent core must then take place via a diffusion process that can be much slower than the rate at which liquid is applied to the core. Gel blocking thus can result in leakage from the absorbent article well before the absorbent core is fully saturated.

Due to the relative expense of superabsorbent materials, various attempts have been made to provide the absorbent garment with “zoned absorbency;” providing those portions of the garment that are most likely to require absorbent capacity with greater concentrations of superabsorbent material. U.S. Pat. No. 5,248,524 to Soderlund, which is incorporated herein by reference, discusses various attempts to provide zoned absorbency. Generally, prior attempts to provide zoned absorbency have concentrated on providing greater or lesser densities of superabsorbent in various parts of a fibrous matrix that has a generally uniform density. While many of the known zoning systems have provided acceptable products, they have been subject to undesirable product variability due to uncontrolled variations in the forming process. For example systems using pulsed (i.e., intermittently activated) superabsorbent particle depositing devices have complicated airflow, hysteresis, timing and momentum problems that may be difficult to predict or control, particularly when production rates increase.

In some cases, providing zoned absorbency may increase the likelihood that gel blocking will occur in an absorbent garment. This may be the case when greater concentrations of superabsorbent are desired to be positioned at the garment's insult point.

These are just a few of the disadvantages of the known absorbent articles and absorbent materials that the preferred embodiments seek to address. The foregoing description of certain materials, methods and systems with their attendant disadvantages in no way is meant to infer that the present invention excludes such materials, methods, and systems. Indeed, certain embodiments of the invention may solve some of the aforementioned disadvantages and other disadvantages, yet may utilize the same or similar materials, methods and/or systems.

### SUMMARY OF THE INVENTION

There exists a need to provide an absorbent core structure and method of making such a structure that provides greater control over the location of high absorbency zones. There also exists a need to provide high absorbency zones having a reduced susceptibility to gel blocking.

The features of the invention generally may be achieved by an absorbent core having a longitudinal dimension, a lateral dimension, and a fibrous matrix with superabsorbent particles distributed within the fibrous matrix. The absorbent core has a folded zone that has at least one laterally-extending fold. The folded zone extends through at least a portion of the longitudinal dimension of the absorbent core.

In one embodiment, the folded zone extends through substantially the entire lateral dimension of the absorbent core.

In another embodiment, a first tissue layer is disposed on a first side of the fibrous matrix, and a second tissue layer is disposed on a second side of the fibrous matrix. The first tissue layer and the second tissue layer may be portions of a single tissue sheet.

In still another embodiment, the folded zone comprises two or more laterally-extending folds. A laterally-extending void may be located between each adjacent pair of laterally extending folds.

In various other embodiments, the fibrous matrix may include tow fibers, which may be cellulose acetate tow fibers, and the superabsorbent particles and fibrous matrix may be a uniform basis weight fibrous matrix/superabsorbent polymer mixture. In another embodiment, the folded zone has greater absorbent properties than other portions of the absorbent core.

In still other embodiments, the absorbent core may be disposed between the topsheet and backsheet of an absorbent garment, and the folded zone may be positioned proximal to an insult point on the topsheet, where exudates from an intended wearer initially strike the topsheet when the garment is worn by the intended wearer.

### BRIEF DESCRIPTION OF THE DRAWINGS

FIG. 1 is an isometric view of an absorbent garment having an absorbent core according to a preferred embodiment of the present invention;

FIG. 2 is a sectional view of a portion of the folded zone of the garment of FIG. 1, as shown from reference line A-A;

FIG. 3 is a side view of an exemplary core forming apparatus; and

FIG. 4 is a side view of an embodiment of a preferred folded core forming apparatus.

### DETAILED DESCRIPTION OF THE PREFERRED EMBODIMENTS

As understood herein, “manufacturing line,” “processing line” and “line” refer to any manufacturing or assembly line. Such a processing line may operate substantially non-stop or intermittently, and may move in substantially one direction or may operate in several directions. Manufacturing lines typically comprise a number of devices to process materials in various ways. The various devices may be operated substantially independently of one another, or they may be partially or entirely integrally controlled by a single driving system having a relatively small number of partially or wholly-independent controllers. Such a system may be based on a modular system such as those disclosed in U.S. Pat. Nos. 5,492,591 and 5,383,988, both to Herrmann et al., each of which is incorporated herein by reference in its entirety.

The “machine direction,” as used herein, is the primary direction in which material or parts are traveling through a processing line at any given point. The material moving through the processing line generally originates from the upstream direction and moves in the downstream direction as it is processed. The “cross-machine direction” or “cross direction” is perpendicular to the machine direction and generally parallel to the plane of the material being processed. The cross machine direction generally corresponds to the width of the material being conveyed. The “z-direction” is orthogonal to the plane defined by crossed vectors in the machine direction and cross machine direction, and generally corresponds to the thickness of the material being conveyed.

As used herein, the term “absorbent garment” or “garment” refers to garments that absorb and contain exudates, and more specifically, refers to garments that are placed against or in proximity to the body of a wearer to absorb and contain various exudates discharged from the body. A non-exhaustive list of examples of absorbent garments includes: diapers, diaper covers, disposable diapers, training pants, feminine hygiene products and adult incontinence products. The term garment includes all variations of absorbent garments, including disposable absorbent garments that are intended to be discarded or partially discarded after a single use (i.e., they are not intended to be laundered or otherwise restored or reused) and unitary disposable absorbent garments that have essentially a single structure (i.e., do not require separate manipulative parts such as a diaper cover and insert). Embodiments of the present invention may be used with all classes of absorbent garments, including those described above and others not described herein.

Absorbent garments and diapers may have a number of different constructions. In each of these constructions it is generally the case that an absorbent core is disposed between a liquid pervious, body-facing topsheet, and a liquid impervious, exterior facing backsheet. In some cases, one or both of the topsheet and backsheet may be shaped to form a pant-like garment. In other cases, the topsheet, backsheet and absorbent core may be formed as a discrete assembly that is placed on a main chassis layer and the chassis layer is shaped to form a pant-like garment. The garment may be provided to the consumer in the fully assembled pant-like shape, or may be partially pant-like and require the consumer to take the final steps necessary to form the final pant-like shape, such as by fastening one or more fastener tabs. In the case of training pant-type garments and most adult incontinent products, the garment often is provided fully formed with factory-made side seams and the garment is donned by pulling it up the wearer's legs. In the case of diapers, a caregiver usually wraps the diaper around the wearer's waist and joins the side seams manually by attaching one or more fastener tabs, thereby forming a pant-like structure. Other garments, such as many feminine care products, do not have a pant-like construction, and the present invention may be used with these garments as well. For clarity, the present invention is described herein only with reference to a diaper-type garment in which the topsheet, backsheet and absorbent core are assembled into a structure that forms a pant-like garment when secured on a wearer using fastening devices, although the invention also may be used with any other type of absorbent garment that may benefit from the use or addition of an absorbent core.

For clarity, features that appear in more than one Figure have the same reference number in each Figure.

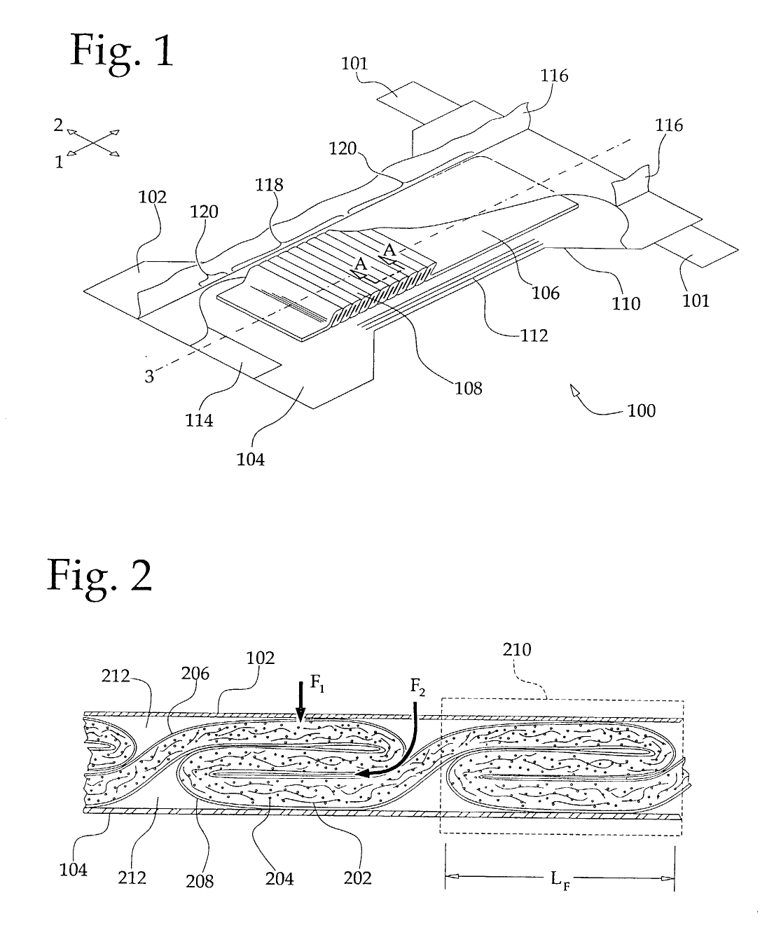

A preferred embodiment of the present invention may be used with a disposable absorbent garment 100 of the diaper type, such as shown, for example, in FIG. 1. The garment 100 of FIG. 1 is shown with the contractile forces of its elastic members removed for clarity in the Figures and the description. The garment 100 chassis is shown having a hourglass shape, but the garment chassis also may have a rectangular shape, a trapezoidal shape, a “T” shape, or any other suitable shape that allows the garment to be affixed to or placed near a wearer. The garment 100 generally has a longitudinal direction 1 corresponding to the front-to-back axis of a wearer, and a lateral direction 2 corresponding to the side-to-side axis of a wearer. The garment generally is symmetrical about a longitudinal centerline 3, but also may have asymmetrical components or shapes.

The garment preferably comprises a topsheet 102, a backsheet 104, which may be either a different size than the topsheet 102 or may be substantially coterminous with the topsheet 102, and an absorbent core 106 disposed between at least portions of the topsheet 102 and the backsheet 104. It should be understood that additional layers may be present between the absorbent core 106 and the topsheet 102 and/or the backsheet 104. Also, additional layers or sheets may be disposed on the topsheet 102 and/or backsheet 104 on the side opposite the absorbent core 106. Such additional layers may be provided to enhance the performance of the garment 100. The dimensions of the additional layers may be the same as or different from the dimensions of the absorbent core 106 and/or topsheet 102 and backsheet 104. Examples of such layers include acquisition layers, transfer layers, wicking layers, storage layers, fluid handling layers, rewet barriers, and the like. These and other useful layers are generally known in the art, and their suitability for use with the present invention will be apparent to those skilled in the art based on the teachings herein, and the invention encompasses all types of additional layers.

A pair of leg cutouts 110 may extend along either side of the garment 100 to provide the garment 100 with a better fit on the wearer. In addition, one or more sets of leg elastics 112 may be disposed to extend along the leg cutouts 110 to contract the leg cutouts around the wearer's legs. The garment 100 further may include a waist elastic system 114, for contracting the garment 100 around the wearer's waist, and a pair of fastener tabs 101 to hold the garment 100 on the wearer. Waste containment systems also may be incorporated into the garment 100. Examples of waste containment systems include such devices as pockets and waste containment flaps 116 (also known as unitary leg gathers or standing leg gathers).

A variety of backsheet and topsheet constructions and materials are available and known in the art, and the invention is not intended to be limited to any specific materials or constructions of these components. The backsheet 104 may be made from any suitable pliable liquid-impervious material known in the art. Typical backsheet materials include films of polyethylene, polypropylene, polyester, nylon, and polyvinyl chloride and blends of these materials. For example, the backsheet may comprise a pigmented polyethylene film having a thickness in the range of 0.02-0.04 mm. In some cases, it may be desirable to provide a partially- or wholly-gas pervious backsheet 104 to encourage airflow in the garment 100 to reduce discomfort, skin rashes and bacterial infections. For example, all or part of the backsheet 104 (or other parts of the garment 100) may be fabricated from a porous film, such as that disclosed in U.S. Pat. No. 6,258,196 to Suzuki et al., which is incorporated herein by reference in its entirety. In addition, to improve the look and feel of the garment 100, the backsheet 104 may be covered with a fibrous, nonwoven fabric layer (not shown) such as is disclosed, for example, in U.S. Pat. No. 4,646,362, which is incorporated herein by reference in its entirety.

The backsheet 104 may comprise a laminate and/or multiple panels of material, such as three panels wherein a central poly backsheet panel is positioned adjacent the absorbent core while outboard non-woven breathable side backsheet panels are attached to the side edges of the central poly backsheet panel. The backsheet also may be formed from microporous poly coverstock for added breathability. The backsheet may further be treated to render all or part of it hydrophilic or hydrophobic, as desired, and may have one or more visual indicators associated with it, such as labels indicating the front or back of the garment, wetness indicators or other characters or colorations. The present invention is not limited to any particular backsheet 104 material or construction.

The moisture-pervious topsheet 102 may comprise any suitable relatively liquid-pervious material known in the art that permits sufficient passage of liquid therethrough. Non-woven topsheet materials are exemplary because such materials readily allow the passage of liquids to the underlying absorbent core 106. Examples of suitable topsheet materials include non-woven spunbond or carded webs of polypropylene, polyethylene, nylon, polyester and blends of these materials.

The topsheet 102 may be formed from one or more panels of material and may comprise a laminated sheet construction. For example, in an embodiment in which multi-panel construction is used, a three-panel topsheet may comprise a central topsheet panel extending along the length of the garment 100, and outer topsheet panels positioned laterally outside of the central topsheet panel. The central topsheet panel preferably is formed from a liquid-pervious material that is either hydrophobic or hydrophilic. The central topsheet panel may be made from any number of materials, including synthetic fibers (e.g., polypropylene or polyester fibers), natural fibers (e.g., wood or cellulose), apertured plastic films, reticulated foams and porous foams to name a few. One preferred material for a central topsheet panel is a cover stock of single ply non-woven material which may be made of carded fibers, either adhesively or thermally bonded, perforated plastic film, spunbonded fibers, or water entangled fibers, which generally weigh from 0.3-0.7 oz./yd² and have appropriate and effective machine direction and cross-machine direction strength suitable for use as an absorbent garment cover stock material, as are known in the art. The outer topsheet panels of a multi-panel construction preferably are substantially liquid-impervious and hydrophobic, preferably at least in the crotch area. The outer edges of the outer topsheet panels may substantially follow the corresponding outer perimeter of the backsheet 104. The outer topsheet panels preferably comprise polypropylene and may be woven, non-woven, spunbonded, carded or the like, depending on the application.

The backsheet 104 and the topsheet 102 preferably are “associated” with one another. The term “associated” encompasses configurations whereby the topsheet 102 is directly joined to the backsheet 104 by affixing the topsheet 102 directly to the backsheet 104, and configurations whereby the topsheet 102 is indirectly joined to the backsheet 104 by affixing the topsheet 102 to intermediate members which in turn are affixed to the backsheet 104. Various bonding methods or combinations of methods may be used to join the topsheet 102 backsheet 104, and any of the other parts comprising the garment 100. Exemplary bonding methods include: adhesive bonding, ultrasonic bonding, heat bonding, chemical bonding, autogenous bonding, and the like.

The garment 100 preferably is fastened onto a wearer by one or more, and preferably two, fastener tabs 101. The fastener tabs 101 preferably are affixed to the chassis of the garment 100 to extend laterally outward (i.e., in the lateral direction 2) from a waist region of the garment, preferably the waist region corresponding to the rear of a wearer when the garment 100 is donned. The fastener tabs 101 may be attached to any part of the garment chassis, such as the topsheet 102, backsheet 104, outer covering, as specially adapted fastening patch or other layer or part of the garment 100. The fastener tabs 101 also may be attached to either side of the garment's chassis, to multiple layers of the chassis, or may be sandwiched between the various sheets comprising the chassis of the garment 100. Variations on the number, location, and attachment configuration of the fastener tabs 101 and other securement devices will be apparent to those skilled in the art based on the teachings herein, and all such variations are within the scope of the present invention. Exemplary fastener tabs include those described in U.S. Pat. Nos. 3,800,796 to Jacob, 4,552,560 to Tritsch, 5,545,159 to Lancaster et al. and 5,685,873 to Bruemmer, each of which is incorporated herein by reference in its entirety. The present invention is not intended to be limited to the use of any particular fastener tabs 101 or to the use of fastener tabs 101 at all.

A waste containment system, such as waste containment flaps 116, may be integrated into the garment 100 to provide an additional physical barrier to the movement or leakage of exudates. Waste containment flaps 116 (also known as unitary leg gathers or standing leg gathers) preferably extend in the longitudinal direction 1 throughout all or part of the longitudinal extent of the garment 100 along opposite sides of the garment's longitudinal center line 3. Other flaps may be positioned in parallel to waste containment flaps 116, or may be positioned to extend across the width of the garment 100 (i.e., in the lateral direction 2) at or near either end of the garment 100 to inhibit the longitudinal flow of exudates.

The waste containment flaps 116 preferably include an elastic material that contracts the flaps around the wearer's body during use. Suitable elastics include elastomeric films, scrims, ribbons, strands, and elastic laminates. The waste containment flaps 116 may be formed from portions of the topsheet 102 and/or backsheet 104, or may be separate assemblies that are attached to the topsheet 102 and/or backsheet 104. The waste containment flaps 116 may be treated with a suitable surfactant to modify their hydrophobicity/hydrophilicity or imbued with skin wellness products as desired. Various other configurations of topsheets 102, backsheets 104 and waste containment systems, such as flaps 116, are known in the art, and the present invention is not intended to be limited to any particular design for these components, or to the use of a waste containment system at all. Exemplary waste containment systems are disclosed in U.S. Pat. Nos. 5,246,431 to Minetola et al., 5,403,301 to Huffmann et al., and 6,123,694 to Pieniak et al. (flaps), U.S. Pat. No. 6,077,254 to Silwanowicz et al. (pockets), and U.S. Pat. No. 6,222,092 to Hansen et al. (flow impediment structures on the topsheet). Each of these disclosures is incorporated herein by reference in its entirety.

Waist elastics 114 may be provided at or near one or both longitudinal ends of the garment 100. The waist elastics 114 may be the same or different at each end of the garment 100 to impart similar or different elastic characteristics to the front and back waist portions of the garment 100. Preferably, the waist elastics 114 comprise elastically extensible foam strips. The foam strips preferably are about 0.50 inches to about 1.50 inches wide and about 3 inches to about 6 inches long. The foam strips preferably are secured between the topsheet 102 and the backsheet 104 by adhesives, heat bonding, ultrasonic bonding, or any other suitable bonding method. The foam strips preferably are polyurethane, but could be any other suitable material that preferably decreases waist band roll over, reduces leakage from the waist ends of the absorbent garment, and generally improves comfort and fit. The front and back waist elastics 114 preferably are stretched to about 150% to about 250% of their unstretched length (in the lateral direction 2), and most preferably to about 200% of their unstretched length, before being adhesively secured between the backsheet 104 and topsheet 102. Alternatively, a plurality of elastic strands or a patch of elastomeric film or scrim material may be employed as waist elastics 114 rather than foam strips. Waist elastics are known in the art, and disclosed, for example, in U.S. Pat. No. 4,430,086 to Repke, which is incorporated herein by reference in its entirety. The present invention is not limited to the use of a particular waist elastic system, or to the inclusion of waist elastics 114 at all.

Leg elastics 112 may be provided along either side of the garment to contract the leg openings around the wearer's legs. In a preferred embodiment the leg elastics 112 comprise three elastic strands on each side of the garment 100, the strands being positioned between the topsheet 102 and the backsheet 114 and extending adjacent each leg cutout 110. Various commercially available materials may be used for the leg elastics 112 and other elastic members that may be incorporated into the garment 100, such as natural rubber, butyl rubber or other synthetic rubber, urethane, elastomeric materials such as spandex, which is marketed under various names, including LYCRA (DuPont), GLOSPAN (Globe) and SYSTEM 7000 (FULFLEX), and so on. The leg elastics 112 may be ultrasonically bonded, heat/pressure sealed using a variety of bonding patterns, glued to the garment 100, or affixed using any other known or later developed method or combination of methods. The present invention is not limited to any particular elastic material, shape, size or number of elastics, or elastic joining method. The selection of appropriate leg elastics 112 and the construction of leg elastic containment systems is known in the art, and disclosed, for example, in U.S. Pat. Nos. 4,573,991 to Pieniak et al., 4,626,305 to Suzuki et al. and 5,660,664 to Herrmann, each of which is incorporated herein by reference in its entirety.

An absorbent core 106 is provided beneath the topsheet 102 to absorb and contain body exudates. Although the absorbent core 106 depicted in FIG. 1 has a substantially rectangular shape, other shapes may be used, such as a “T” shape or an hourglass shape. The absorbent core 106 may extend into either or both of the garment's waist regions, or may be located primarily in the crotch. The absorbent core 106 also may comprise a number of layers of similar or different construction. The absorbent core 106 may be attached to or captured between the topsheet 102 and backsheet 104, or maybe joined to the garment by any other suitable method, as will be appreciated by those skilled in the art.

The absorbent core 106 may be made from any absorbent material or materials, or combinations of such materials, known in the art or hereafter discovered. Absorbent core materials are known in the art and exemplary core materials are disclosed, for example, in U.S. Pat. Nos. 4,610,678 to Weisman et al., 5,246,429 to Poccia et al., 5,137,537 to Herron et al., 5,147,345 to Young et al., 5,281,207 to Chmielewski et al., 6,068,620 to Chmielewski, and U.S. Statutory Invention Registration No. H1,565 to Brodof et al., each of which is incorporated herein by reference in its entirety. Preferably the absorbent core 106 comprises a combination of a porous fibrous web and superabsorbent particles. The absorbent core 106 may be chosen to absorb particular fluids or to absorb fluids generally. The absorbent core 106 preferably is thin in order to improve the comfort and appearance of a garment 100 containing the absorbent core 106.

Referring now to FIG. 2, a preferred absorbent core 106 comprises a fibrous matrix 202 into which superabsorbent particles (SAP) 204 have been dispersed. The fibrous matrix 202 and SAP 204 preferably are partially or wholly contained within tissue layers 206, 208. In other embodiments, however the tissue layers 206, 208 may be omitted.

The fibrous matrix 202 may comprise any suitable absorbent core material. Exemplary materials for the fibrous matrix 202 include cellulosic fibers such as wood pulp fluff or fluffed bleached kraft softwood pulp, cotton, cotton linters, rayon, fibrous absorbent gelling materials, cellulose acetate, synthetic polymeric fibers, and the like. Such materials are known in the art and disclosed, for example, in U.S. Pat. No. 4,610,478 to Weisman et al., which is incorporated herein by reference in its entirety. The fibrous matrix 202 also may comprise foam material. Preferably, the fibers or other material comprising the fibrous matrix 202 are hydrophilic. Other suitable absorbent core materials, as are known in the art or later discovered, also may be used.

In a preferred embodiment, the fibrous matrix 202 comprises a relatively low density matrix of opened tow fibers. Tow fibers are relatively long fibers of material that are bundled together to form a continuous web of material, known as a tow web. The tow web may be stored in a compact form, then “opened” (i.e., fluffed or bloomed) into a cotton-like form before integration into the absorbent core 106. Various devices may be used to open the tow web, such as mechanical agitators and high velocity air streams, such as those disclosed in U.S. Pat. Nos. 5,331,976 to St. Pierre, and 4,525,385 to Pryor, each of which is incorporated herein by reference in its entirety. The relatively long fibers of the tow impart additional tensile strength to the absorbent core 106, allowing less tow material to be used to make a stable absorbent core 106 that will not disintegrate during manufacture or use. The resulting absorbent cores 106 typically have a lower overall density than other types of core that provide an equivalent amount of absorbent capacity, and are relatively thin and light. Absorbent cores 106 using such materials are referred to herein as “tow-based” cores. The construction and use of such tow-based cores are disclosed generally in U.S. Pat. No. 6,068,620 to Chmielewski, and U.S. Statutory Invention Registration No. H1,565 to Brodof et al., each of which is incorporated herein by reference in its entirety.

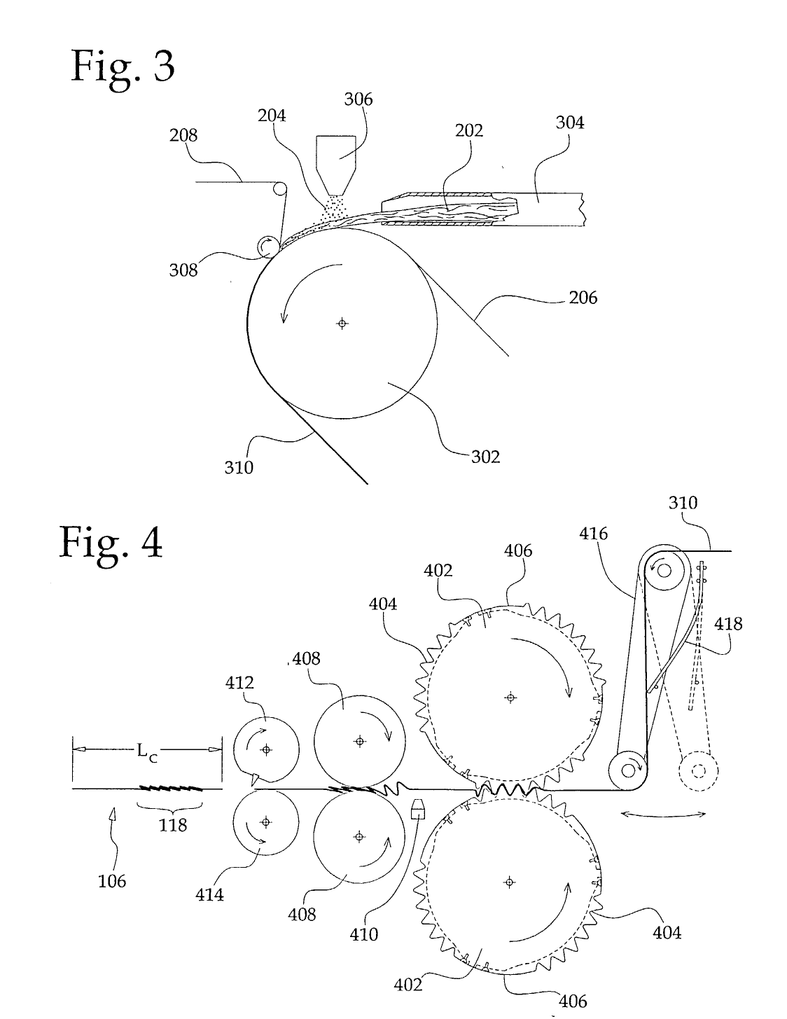

A preferred process for manufacturing a tow-based core is shown in FIG. 3. In the process of FIG. 3, a first tissue layer 206 is provided (as a continuous supply) to a rotating drum 302, and a fibrous matrix 202 of opened tow material is applied on top of the first tissue layer 206. Superabsorbent material 204 is then deposited onto the fibrous matrix 202 and first tissue layer 206, and a second tissue layer (in the form of a continuous supply) is placed on top of the fibrous matrix 202 and SAP 204 by a press roll 308 to form a continuous core supply 310. The first and second tissue layers 206, 208 may be adhesively-, ultrasonically-, heat-, or otherwise bonded to one another and to the fibrous matrix 202 and/or SAP 204. Adhesive may be applied to the first and/or second tissue layer 206, 208 prior to forming the tow-based core. The fibrous matrix 202 preferably is provided from a forming chamber 304 that uses compressed air or a mechanical means to open a tow web into a fluffed form.

The SAP 204 preferably is provided by a depositing mechanism 306 that meters out SAP 204 at the desired rate, which may be controlled to vary appropriately, as the speed of the operation increases or decreases, to provide the desired SAP distribution in the fibrous matrix 202. Any particulate matter feeding system may be used as the depositing mechanism 306 so long as it is capable of providing SAP 204 at the desired flow rate. Exemplary depositing mechanisms 306 include auger-type feeders, such as those available from SolidsFlow Corporation of Fort Mill, S.C. (which may use a loss-in-weight metering system or other types of metering systems), pneumatic feed systems that use a stream of SAP particles entrained in an air flow, and the like. Other processes also may be used for forming a tow-based core, such as the processes described in U.S. Pat. No. 6,068,620 and U.S. Statutory Invention Registration H1,565. The present invention is not intended to be limited to any particular process for forming a tow-based absorbent core 106.

Regardless of whether a tow-based construction, a conventional fluff construction or other construction is used, the absorbent cores 106 preferably are provided with a substantially uniform amount of SAP 204 per unit length of the fibrous matrix 202, as they are being produced, to create a uniform basis weight fibrous matrix/SAP mixture. In a uniform basis weight fibrous matrix/SAP mixture, the amount of SAP per unit length or per unit weight of the fibrous matrix does not vary by more than about 50% throughout the unfolded length of the absorbent core 106, and more preferably does not vary by more than about 35% throughout the unfolded length of the absorbent core 106, and most preferably does not vary by more than about 20% throughout the unfolded length of the absorbent core 106. In other embodiments, however, the absorbent cores may be produced having a zoned SAP distribution, in which the amount of SAP 204 substantially varies along the length or width of the absorbent core 106. It will be understood that, in some instances, during further processing and use the SAP may migrate through the fibrous matrix 202. This SAP migration may ultimately lead to a relatively disordered SAP distribution or to local high or low concentrations of SAP 204, even in absorbent cores 106 that were originally produced with a uniform basis weight fibrous matrix/SAP mixture.

In other preferred embodiments, the SAP 204 may be omitted from the absorbent core 106. Such an embodiment may be desirable, for example, in products intended to be used in aqueous environments, such as while the wearer is swimming or otherwise submerged or exposed to substantial amounts of environmental moisture.

Certain fibrous materials preferably are used to form the fibrous matrix 202 of a tow-based absorbent core 106 of the present invention. Preferably, the tow materials maintain high SAP efficiencies, even when the SAP concentration is relatively high. Preferred tow materials include cellulose esters, such as cellulose acetate, cellulose propionate, cellulose butyrate, cellulose caproate, cellulose caprylate, cellulose stearate, highly acetylated derivatives thereof such as cellulose diacetate, cellulose triacetate and cellulose tricaproate, and mixtures thereof such as cellulose acetate butyrate. A suitable cellulose ester preferably has some ability to absorb moisture (but absorptive capability is not necessarily required), preferably is biodegradable, and is influenced not only by the substituent groups but also by the degree of substitution. The relationship between substituent groups, degree of substitution and biodegradability is discussed in W. G. Glasser et al., BIOTECHNOLOGY PROGRESS, vol. 10, pp. 214-219 (1994), the disclosure of which is incorporated herein by reference in its entirety. Other suitable fibrous materials include rayon fibers, Courtauld's LYOCELL fibers, polyacrylonitrile fibers, surface-modified (hydrophilic) polyester fibers, surface-modified polyolefin/polyester bicomponent fibers, surface-modified polyester/polyester bicomponent fibers, cotton fibers, blends of the foregoing materials, and the like.

Of the foregoing, cellulose acetate tow fibers are the most preferred materials for use as the fibrous matrix 202 of a tow-based absorbent core 106. Cellulose acetate has been found to provide high SAP efficiencies, even when relatively high SAP concentrations are used, and is moisture-absorbent and biodegradable. Preferably, the denier per fiber (dpf) of each cellulose acetate fiber will be in the range of about 1 to 9, preferably about 3 to 6, and most preferably about 4. For the same weight product, filaments of lower dpf may provide increased surface area and increased moisture absorption. The total denier of the tow may vary within the range of about 20,000 to 60,000, depending upon the process used, and preferably is about 35,000. The fibers may have a circular, ovate, rectilinear, or any other cross section. In one preferred embodiment, the fibers have a tri-lobal cross section with an area of about 3.36×10⁻⁶ cm². Such a cross-sectional shape may provide improved bending stiffness, increased wicking, or other beneficial properties.

Also in a preferred embodiment, the tow has crimped filaments. Crimps aid with opening the tow, increase the available filament surface area for superabsorbent material immobilization and increase moisture absorption. It is anticipated that gel blocking also may be reduced by using crimped tow in the absorbent core 106. As therefore may be understood, more crimp is typically better, with an excess of about 20 crimps per inch being preferred. Continuous filament cellulose acetate tow having crimped filaments with about 25 to 40 crimps per inch is commercially available from Hoechst Celanese Corporation of Charlotte, N.C.

If desired, an absorbent core 106 of multiple layer thickness may be provided. To this end, the tow may be, for example, lapped or crosslapped in accordance with conventional procedures. In this way, a superabsorbent, absorptive material of a desired weight and/or thickness may be provided. The specific weight or thickness will depend upon factors including the particular end use.

Any superabsorbent particles (SAP) 204 now known or later discovered may be used in the absorbent core 106 (whether of conventional construction, tow-based construction or other construction), so long as it is capable of absorbing liquids. Useful SAP materials are those that generally are water-insoluble but water-swellable polymeric substances capable of absorbing water in an amount that is at least ten times the weight of the substance in its dry form. In one type of SAP, the particles or fibers may be described chemically as having a backbone of natural or synthetic polymers with hydrophilic groups or polymers containing hydrophilic groups being chemically bonded to the backbone or in intimate admixture therewith. Included in this class of materials are such modified polymers as sodium neutralized cross-linked polyacrylates and polysaccharides including, for example, cellulose and starch and regenerated cellulose which are modified to be carboxylated, phosphonoalkylated, sulphoxylated or phosphorylated, causing the SAP to be highly hydrophilic. Also included are water swellable polymers of water soluble acrylic or vinyl monomers crosslinked with a polyfunctional reactant. Such modified polymers also may be cross-linked to reduce their water-solubility, and such cross-linked SAPs have been found to provide superior performance in some absorbent cores. A more detailed recitation of superabsorbent polymers is found in U.S. Pat. No. 4,990,541 to Nielsen, which is incorporated herein by reference in its entirety.

The SAP preferably is selected to provide high absorbency performance for the particular application. The measure of the SAP's absorbency performance may be evaluated in a number of ways, as will be understood by those skilled in the art, and preferred SAPs 204 may be selected based on one or more of these evaluation criteria. For example, it may be desirable to provide a SAP having a high measure of saline flow conductivity (SFC), as is described in U.S. Pat. No. 5,562,646 to Goldman et. al, which is incorporated herein by reference in its entirety. In all cases, it is preferred that the SAP be fully, rapidly and efficiently utilized while in the absorbent core; that is, the SAP 204 should be provided in sufficient quantity and have sufficient properties to absorb all of the fluid exudates introduced to the garment 100 without having leakage caused by gel blocking or other inhibitions to the imbibition of fluid, but should not be present in great excess of the required amount.

Commercially available SAPs include a starch modified superabsorbent polymer available under the trade name SANWET® from Hoechst Celanese Corporation, Portsmouth, Va. SANWET® is a starch grafted polyacrylate sodium salt. Other commercially available SAPs include a superabsorbent derived from polypropenoic acid, available under the trade name DRYTECH® 520 SUPERABSORBENT POLYMER from The Dow Chemical Company, Midland Mich.; AQUA KEEP manufactured by Seitetsu Kagaku Co., Ltd.; ARASORB manufactured by Arakawa Chemical (U.S.A.) Inc.; ARIDALL 1125 manufactured by Chemdall Corporation; and FAVOR manufactured by Stockhausen Inc. Still other commercially available SAPs include SA55SX, available from Sumitomo Chemical Co. Ltd. of Osaka, Japan, and T7700 and T7200 and other SAP provided by BASF of Mount Olive, N.J.

The SAP may be provided in any particle size, and suitable particle sizes vary greatly depending on the ultimate properties desired. Preferably, a fine particulate rather than a coarse particulate, is used in the invention, and preferably a fine particulate that passes through an about 200 mesh screen is used.

It has been known to prepare absorbent cores comprising cellulose acetate tow or other polymeric fibers and SAP, as described in U.S. Statutory Invention Registration H1565, and U.S. Pat. Nos. 5,436,066 to Chen, and 5,350,370 to Jackson et al., each of which is incorporated by reference herein in its entirety. It was conventional to add tackifying agents, specific size fibers, or specific fibers in combination with fluff, in order to prepare the absorbent core and immobilize the SAP particles. These additional materials may add to the density of the core, or otherwise adversely affect the overall performance of the absorbent garment made therefrom. Thus, it is preferred not to use ethylene glycol, tackifying agents, and very small particulate fibers in the invention, although they may be used to the extent they do not unduly reduce the overall performance of the absorbent core 106 of the present invention.

The amount of SAP 204 provided to the absorbent core 106 may vary depending on the absorbent properties of the SAP 204 and the particular application for which the absorbent core 106 is being prepared. In various embodiments, the SAP concentration may be as high as 95% or more (by weight) of the combined weight of the fibrous matrix 202 and the SAP 204. In those embodiments having a tow-based construction, the SAP 204 concentration generally may be higher than those using conventional constructions because the relatively strong tow material allows the construction of a lighter fibrous matrix 202 for a desired weight of SAP 204 than is possible or practical using other types of fibrous matrix 202.

Additional particles or fibrous additives may be added to the fibrous matrix 202 of regular absorbent cores or tow-based absorbent cores to help maintain high SAP efficiency, to reduce the cost of the garment, or to provide other benefits. In one embodiment, for example, about 1-10%, and preferably about 5%, by weight of thermally bondable synthetic fibers may be added to the fibrous matrix 202 to impart additional wet strength to the laminate. These additive fibers may improve the stability of the absorbent core 106 during use of the diaper. The preferred synthetic fibers for such an embodiment are polyolefin/polyester fibers and polyester/polyester bicomponent fibers.

In a tow-based core, the fibrous matrix 202 may comprise a combination of preferred tow materials, such as a blend of cellulose ester and conventional soft or hard wood fibers. Such combinations may be useful to maintain the improved SAP efficiency available from a crimped filament tow-based absorbent core 106 while providing additional benefits. For example, it has been discovered that an absorbent core 106 having a 150 g/m² composite comprised of 80% SAP, 10% cellulose acetate, and 10% conventional fluff pulp has a SAP efficiency of about 85%, whereas an absorbent core 106 comprised of 80% SAP and 20% fluff pulp SAP has a SAP efficiency of about 70%.

Preferred particulate additives that may be added to the absorbent core 106 (whether conventional or tow-based) may comprise insoluble, hydrophilic polymers with particle diameters of 100 μm or less. These particulate additives may be chosen to impart optimal separation of the SAP particles 204. Examples of preferred particulate additive materials include, but are not limited to, potato, corn, wheat, and rice starches. Partially cooked or chemically modified (i.e., modifying hydrophobicity, hydrophilicity, softness, hardness, etc.) starches also may be effective. Most preferably, the particulate additives comprise partially cooked corn or wheat starch because in this state, the corn or wheat are rendered larger than uncooked starch and in the cooked state remain harder than even swollen SAP. In any event, regardless of the particulate additive chosen, one of the many important criteria is to use particulate additives that are hard hydrophilic materials relative to swollen SAP or which are organic or inorganic polymeric materials about 100 microns in diameter. Fibrous and particulate additives can be used together in these absorbent laminates. Examples of SAP/particulate and SAP/fiber/particulate additives include those described in, for example, U.S. Pat. No. 6,068,620.

Other particulate or powdered additives also may be deposited within the absorbent core 106 to provide odor control, skin wellness, and improved appearance. For example, zeolites, sodium bicarbonate and perfumes may be added to the fibrous matrix 202 or the tissue layers 206, 208 to reduce or mask odors, and titanium dioxide or other color-imbuing compounds may be added to provide the absorbent core 106 with a more pleasant color.

The fibrous matrix 202 and SAP 204 preferably are partially or wholly contained within tissue layers 206, 208. The tissue layers 206, 208 preferably comprise a low basis weight fibrous material having sufficient wet and dry strengths to contain the fibrous matrix 202 and SAP 204 during manufacture and use of the garment 100, sufficient permeability to allow relatively unimpeded passage of fluids and other exudates therethrough, and sufficient pore size to prevent substantial migration of SAP therethrough. The tissue layers 206, 208 may comprise any material, such as a crepe-wadding, forming tissue or barrier tissue manufactured from a batt of wood pulp fluff fibers, or synthetic materials, and may be treated with a surfactant to render them hydrophilic. The use and selection of tissue layers 206, 208 are generally known in the art, and a skilled artisan will be able to employ suitable tissue layers 206, 208 without undue experimentation based on the teachings herein.

In a preferred embodiment, the tissue layers 206, 208 comprise two separate layers that encase the absorbent core 106. The tissue layers 206, 208 optionally also may encase one or more additional layers, as noted elsewhere herein. Preferably, a first tissue layer 206 is located generally between the topsheet 102 and the absorbent core 106. A second tissue layer 208 preferably is located between the backsheet 104 and the absorbent core 106. Also in a preferred embodiment, the first tissue layer 206 is hydrophilic and fluid pervious, and the second tissue layer 208 is hydrophobic and fluid impervious, although both tissue layers 206, 208 may have similar properties. The tissue layers 206, 208 also may comprise separate portions of a single tissue sheet that has been folded to encase the absorbent core 106. In such a case, the portion of the tissue sheet that forms the second tissue layer 208 may be zone treated to render it hydrophobic and fluid impervious. The perimeter of the tissue layers 206, 208 may be crimped, folded, sealed or bonded to further help contain the fibrous matrix 202 and SAP 204.

In one embodiment, portions of the fibrous matrix 202 and SAP 204 of the absorbent core 106 may be adhesively or thermally bonded to improve the absorbent core's wet strength and core stability. Such bonding, however, may result in reduced absorption rates and SAP efficiency. In another embodiment the SAP 204 and fibrous matrix 202 may be hydrogen bonded to one or both of the tissue layers 206, 208. It has been found that when a tow-based fibrous matrix 202 having a high concentration of SAP 204 is hydrogen bonded to first and second tissue layers 206, 208 to form an absorbent core 106, the SAP efficiency is not impaired, wet strength increases, and the first and second tissue layers 206, 208 add stability to the core 106 during manufacture. This performance improvement is believed to be the result of beneficial liquid distribution provided by the intimate bond between the fibers of the fibrous matrix 202 and the tissue layers 206, 208.

In another preferred embodiment, the first and second tissue layers 206, 208 may be coated with adhesive prior to being placed on either side of the absorbent core 106, thereby providing strength to the absorbent core 106 and adhesively holding a portion of the SAP 204 in place during use. The tissue layers 206, 208 may be provided having a width greater than the fibrous matrix 202, and the portions of the tissue layers 206, 208 extending past either side of the fibrous matrix 202 may be bonded to one another to provide further SAP and fiber retention capability. In still another embodiment, the fibrous matrix 202 may be provided having about 1-5% by weight of thermally bondable synthetic fibers, which may be heated to thermally bond the fibrous matrix 202 to the tissue layers 206, 208.

The absorbent core 106 preferably is formed using a dry process, however wet processes or other processes also may be used to form the absorbent core 106. Dry processes are believed to have numerous benefits over wet processes (which sometimes are called “wet-laid” processes). In wet processes, the material that eventually forms the fibrous matrix 202 typically is immersed in a fluid having superabsorbent particles 204 mixed or suspended therein, and may require additional drying steps and other steps that add to the complexity and cost of the core forming process. In addition, wet processes often require the absorbent core to be manufactured off of the main assembly line. An exemplary wet-forming processes is disclosed in U.S. Pat. No. 5,997,690 to Woodrum, which is incorporated herein by reference in its entirety. Dry processes typically have lower operating costs than wet processes because the equipment used in dry processes typically is less complex and can run at higher line speeds. Other advantages also may be realized by the use of a dry process, as will be understood by those skilled in the art. Furthermore, dry forming processes may often be adapted for use directly on the line of conventional diaper machines. As such, the absorbent cores 106 of a preferred embodiment of the present are manufactured by a dry forming process.

In an embodiment of the invention in which the absorbent core 106 is a dry formed tow-based core, the total basis weight of the absorbent core 106, including the fibrous matrix 202, SAP 204, tissue 206, 208, and any additional layers and/or additives, preferably may be from about 100 grams per square meter (gsm) to about 1,000 gsm. Most preferably, the total basis weight of such an absorbent core 106 is about 500 gsm to about 700 gsm.

The absorbent core 106 of the invention preferably is provided with zones having relatively high absorbent capacity by folding portions of the absorbent core 106 over on itself to increase the amount of fibrous matrix 202 and SAP 204 present in those zones. These high-capacity folded zones 118 preferably are positioned adjacent insult points where body fluids are most likely to strike the garment 100, but also may be located in other regions of the absorbent core 106, such as in regions where fluids are likely to settle during use. Exemplary insult points are located in the front half of the garment's crotch (for male wearers) and in the longitudinal center of the crotch (for female wearers). The folded zone or zones 118 also may be positioned to impede the migration of bowel movement within the garment 100. An unfolded zone or zones 120, where the garment is not folded onto itself, preferably is positioned where less absorbent capacity is likely to be required. The present invention may be adapted to have a single folded zone 118 or a number of separate folded zones 118. An absorbent core 106 of the present invention also may have a single folded zone 118 and no unfolded zones 120. In addition, absorbent garments 100 incorporating a folded absorbent core of the present invention may be produced for gender-specific applications to accommodate the specific absorbency zoning requirements of male and female wearers. For example, folded zones 118 may be positioned in or towards the front half of a garment 100 intended for male wearers. Other considerations regarding the desirable location of locations for the zoned absorbency provided by the present invention are disclosed, for example, in U.S. Pat. Nos. 4,333,463 to Holtman, 4,685,915 to Hasse et al., and 5,009,650 to Bernardin, each of which is incorporated herein by reference in its entirety. The present invention is not intended to be limited to any particular location for the folded zone or zones 118 that provide such zoned absorbency.

Each folded zone 118 preferably comprises one or more laterally extending “z” or “s” shaped folds 210 (FIG. 2). Each fold 210 comprises folded portions of the fibrous matrix 202, and preferably also includes the adjacent portions of the tissue layers 206, 208. Each fold 210 has approximately three times the amount of SAP 204 per unit area than the unfolded portion of the absorbent core 106. If greater SAP concentration is desired, the folds may themselves be doubled over, however care must be taken not to make the folded zone 118 excessively bulky, which may impair the garment's comfort or appearance. Laterally extending voids 212 may be provided between each fold 210, as shown in FIG. 2. Alternatively, the folds 210 may be compressed together in the longitudinal direction 1 so that the voids 212 are substantially eliminated. The folds 210 may be held in the folded position by any suitable mechanism. For example the folds 210 may be held in position by being contained between the topsheet 102 and the backsheet 104, by being adhesively or otherwise secured to the topsheet 102 and/or backsheet 104 or by being adhesively or otherwise secured in the folds themselves. If adhesive is used to hold the folds 210, the adhesive preferably is applied at discrete locations to minimize any impediment to fluid flow that the adhesive may cause.

A single, relatively long fold 210 may be used to provide zoned absorbency to the absorbent core 106, but more preferably, a number of shorter folds 210 are provided. The number and length of the folds 210 may be a function of the foldability of the absorbent core 106, the desired size of the folded zone 118, and the desire to obtain improved SAP utilization. It is anticipated that the use of folds 210 will provide greater SAP utilization, because the folds 210 provide passages to the lower portions of the absorbent core 106 (i.e., the portions of the absorbent core 106 that are closer to the backsheet 104, and relatively far, in the z-direction, from the topsheet 102 and the insult point or points). Furthermore, the folds 210 may impede the migration of SAP 204 through the fibrous matrix 202, thereby reducing the incidence of localized zones of relatively high and low SAP concentration when such concentrations are not desired.

The expected improved access to the SAP 204 provided by the present invention is demonstrated by the arrows F₁ and F₂ of FIG. 2, which represent fluid flows into the absorbent core 106. Arrow F₁ designates an anticipated primary flow of fluid directly into the absorbent core 106 as it is expelled from the wearer. The primary flow of fluid into the garment 100 may be facilitated by using a relatively low-density fibrous matrix 202, such as a tow-based fibrous matrix 202. It has been found that tow-based fibrous matrices 202 may allow a faster initial influx of fluid, and greater overall core permeability than conventional fibrous matrices 202 comprised of fluff pulp or similar materials.

Arrow F₂ designates an anticipated secondary flow of fluid into the voids 212, which then moves, by mass flow, diffusion, capillary action or the like, into the lower portion of the absorbent core 106. It is believed that the secondary flow into the voids 212 and the lower portions of the absorbent core 106 will provide improved core permeability, thereby allowing improved core utilization and reduced gel blocking (or a reduction in the negative consequences of any gel blocking that does occur) by providing an additional flow path to supplement the primary flow. The voids 212 also may act as temporary storage reservoirs for fluid that is not immediately absorbed.

It is also anticipated that the folds 210 may act as physical barriers to impede the displacement or relocation of bowel movement contained within the garment 100. In one embodiment, the folds may be sized and positioned to act as “pockets” to help contain bowel movement in a longitudinal direction. Such pocket-like folds may be sealed at their ends (i.e. along the lateral edges of the absorbent core 106) or at other locations to help prevent the lateral displacement of bowel movement.

The secondary flow's penetration into the core's lower portion may be facilitated or improved by using a first tissue layer 206 that has high wicking properties or other beneficial fluid transfer properties that convey the fluid in the longitudinal direction 1. An additional layer of material (not shown), preferably having high longitudinal wicking properties, also may be provided on the first tissue layer 206, either before or after the absorbent core 106 is folded, to provide improved secondary flow penetration. Also, the fold 210 may be constructed to leave gaps between the portions of the first tissue layer 206 that face one another. Other methods of improving the secondary flow penetration into the lower portion of the core will become apparent to those skilled in the art based on the teachings herein and practice of the invention.

The secondary flow penetration also may be adjusted by varying the number, spacing and length of the folds 210. All of the folds 210 of an absorbent core 106 of the present invention may have substantially the same length L_{F} (i.e., the length of the fold in the longitudinal direction 1) and/or spacing (i.e., the distance from the base of one fold 210 to the next). Alternatively, an absorbent core 106 of the present invention may be provided with numerous folds 210 that have various different spacings and lengths L_{F}. It is anticipated that shorter fold lengths L_{F} may allow greater SAP utilization, but may be more difficult to keep in their desired folded-over position during manufacture and use of the garment 100. The minimum fold length L_{F} also may be constrained by the physical properties of the absorbent core 106, the stiffness of which may resist the formation of shorter folds. For this reason, in a particularly preferred embodiment, the absorbent core 106 is a tow-based core. Tow-based cores generally are thinner, less bulky and less dense than cores having conventional fluff pulp or other conventional materials as the fibrous matrix 202. As such, tow-based cores may be produced to be less rigid, and thus better suited to being folded, than other types of absorbent core 106. In addition, the thinness of tow-based cores may allow the folds 210 to have tighter bends without tearing the tissue layers 206, 208 or substantially damaging the fibrous matrix 202. In some cases, however, it may be desirable to permanently compress, break or sever the fibrous matrix 202 at the point at which it is folded to reduce the bulk of the absorbent core 106. In a preferred embodiment, in which the absorbent core 106 is a tow-based core, the folds have a length of about 10 millimeters (mm) to about 300 mm. In another preferred embodiment, the absorbent core 106 is a tow-based core having about 5 to about 15 folds 210, each having a length L_{F} of about 20 mm to about 50 mm.

The secondary flow shown by arrow F₂ also may have a lateral component that assists with redistributing the fluid along the width of the absorbent core 106 to help fully utilize the SAP 204 in the absorbent core's lateral reaches. This lateral component may be adjusted by changing the size of the voids 212, modifying the lateral wicking properties of the topsheet 102 and first tissue layer 206, adding physical flow barriers or by any other methods, as will be apparent to those skilled in the art based on the teachings herein and practice of the invention.

It is preferred that fold 210 extend through substantially the entire lateral dimension of the core, and most preferably fold 210 extends through the entire lateral dimension. Various embodiments of the invention include those in which fold 210 does not extend across the entire lateral dimension, but only 75% or so of the lateral dimension, thereby extending through the entire lateral dimension.

It is anticipated that the additional improved fluid flow characteristics of a tow-based absorbent core of an embodiment of the present invention will allow the use of a variety of different superabsorbent polymer materials. The various different superabsorbent polymers have different properties with respect to their capacity (i.e., the total amount of fluid that can be retained per unit weight of the SAP), permeability (i.e., the degree to which a fluid-swollen SAP particle allows additional fluid to pass or diffuse through it) and absorbency under load (i.e., the SAP's ability to retain fluids under pressure). In some cases, these properties may balance off of one another, for example, a SAP having relatively high absorbency under load and/or total capacity may have relatively low permeability, making such a SAP likely to suffer from gel blocking. The improved fluid permeability of an embodiment of the present invention, particularly an embodiment using a tow-based construction, allows the use of SAP 204 that has relatively high absorbency under load and/or total capacity and relatively low permeability, without suffering from reduced core utilization caused by gel blocking. Other beneficial uses of different SAP 204 materials also may be provided using embodiments of the present invention.

Referring now to FIG. 4, a preferred apparatus and process for manufacturing an absorbent core having folded zones is described. A folded core manufacturing device preferably comprises a pair of counter-rotating folding rolls 402 that provide multiple creases in portions of a continuous core supply 310. The continuous core supply 310 may be fabricated as described with reference to FIG. 3, or may be fabricated by any other suitable methods or means. The present invention is not intended to be limited to any particular method or device for forming the absorbent core supply 310.

In a preferred embodiment, the folding rolls 402 each have sets of teeth 404 between which the continuous core supply 310 passes as the folding rolls 402 rotate. The teeth 404 of each folding roll 402 are shaped and positioned to mesh with the corresponding teeth 404 of the other folding roll 402, and are spaced apart from one another enough to prevent damage to the continuous core supply 310, but are close enough to one another to deform the continuous core supply 310 to have a number of creases or folds. Smooth surfaces 406 are disposed between each set of teeth 404 on the folding rolls 402. The continuous core supply 310 is not folded as it passes between the smooth surfaces 406, which preferably are spaced from the corresponding smooth surface 406 of the opposite folding roll at a distance suitable to grasp and convey the continuous core supply 310 without damaging or over-compressing it.

Other folding devices may be used to provide folds 210 in the continuous absorbent core supply 310. For example, the folds 210 may be formed by ribbed belts, feeding the continuous core supply 310 into a chamber that moves slower than the absorbent core supply 310 (preferably by momentarily accelerating the absorbent core supply 310) to cause the formation of folds, applying alternating jets of compressed air to the absorbent core supply 310, stamping the absorbent core supply 310 between interlocking crossbars, and so on. It also will be understood that the absorbent cores 106 may be folded after being severed from a continuous core supply 310, or may be processed as individual cores from the outset, or may be processed without ever being part of a continuous core supply 310. These and other steps of the core forming process may be manipulated, reordered, omitted, or supplemented without departing from the scope of the present invention, as will be understood by those skilled in the art.

The creased continuous core supply 310 then is conveyed to a pair of press rolls 408 that flatten the creases into folds. The press rolls 408 are spaced from one another to provide the desired amount of compression to the creases, and may be adjustable. Alternatively, press belts, reciprocating presses, or other devices may be used in place of the press rolls, as will be understood by those skilled in the art. Air jets or mechanical guides (not shown) also may be provided to operate in conjunction with the press rolls 408 to initiate the formation of the folds 210 in the desired direction.

An adhesive applicator 410, such as a commercially available CF-200 series CONTROLLED FIBERIZATION™ hot melt adhesive applicator available from Nordson Corporation of Norcross, Georgia, or other applicators 410, may be used to apply an adhesive to one or both sides of the continuous core supply 310. The adhesive may be useful for bonding the folds 210 in place, however it should not be applied such that it will contact the press rolls 408 or other parts of the machinery. Preferably, the adhesive is discretely applied only within the creases so that it is only present between the folds 210.

The folding rolls 402 and press rolls 408 form a continuous supply of core material that has spaced apart folding zones 118. The continuous core supply 310 then is severed into individual absorbent cores 106. In order to manufacture a series of absorbent cores 106 having folding zones 118 in the desired location, a proper selection of the diameter of the folding rolls 402 and positions of the sets of teeth 404 should be made. Generally, the absorbent core length L_{C} may be modified by providing larger or smaller diameter folding rolls 402. The size of the folding zones 118, the number of folds 210, the length of the folds L_{F}, and, to some degree, the spacing between the folded zones 118 may be modified by changing the sets of teeth 404 and smooth surfaces 406 on the folding roll, which may be replaceable as interchangeable modules. The proper selection of these dimensions and locations will depend on the desired shape and size of the absorbent cores being manufactured, and those skilled in the art will be able to properly size and set up the folding rolls 402, teeth 404 and smooth surfaces 406 without undue experimentation.

The individual absorbent cores 106 may be severed from the continuous core supply 310 using any suitable device. In a preferred embodiment, a rotating cut roll 412 having one or more knife edges may be provided to separate individual absorbent cores 106 by pressing the continuous core supply 310 against an anvil roll 414. The speed of the cut roll 412 and/or the diameter of the cut roll 412 may be adjusted or changed to produce absorbent cores 106 having different lengths L_{C}. These and other well known or later developed cutting devices, such as laser cutters, water cutters, and the like, also may be used, as will be understood by those skilled in the art. The present invention is nit intended to be limited to the use of any particular cutting device or to the use of a cutting device at all.

The details of the design of the folding rolls 402, press rolls 408 and cutting roll 412 will depend on the type and dimensions of the particular continuous core supply 310 being processed. Those skilled in the art will be able to provide these and other devices to fold the continuous core supply 310 without undue experimentation based on the teachings herein.

A tension arm 416 or other tensioning device preferably is provided upstream of the folding rolls 402. Assuming that the folding rolls 402 rotate at a substantially constant angular velocity, the teeth 404 will pull the continuous core supply 310 faster than the smooth surfaces 406 do. So as the folding rolls 402 rotate, the continuous core supply 310 will periodically accelerate and decelerate. To accommodate for this change in speed, the tension arm 416, which may have a tensioning spring 418, pivots back and forth to take up slack in the continuous core supply during the slower pull speed periods and to let out slack during the faster pull speed periods. The tensioning spring 418 may comprise any suitable spring or retracting device, such as coil springs, leaf springs, hydraulic and pneumatic rams and the like, and the present invention is not limited to the use of any particular device for the tensioning spring 418. The design and use of tension arms 416 and other tensioning devices, such as moving bars or rollers, festoons and the like, is known in the art, and a skilled artisan will be able to employ such devices with the present invention without undue experimentation based on the teachings herein.

It is anticipated that using folded zones 118 to create zoned absorbency absorbent cores, as provided by the present invention, will provide several benefits over known SAP targeting devices and zoned absorbent cores. For example, the absorbent core 106 may be constructed having a uniform distribution of SAP 204 within the fibrous matrix 202, yet still provide zoned absorbency. As such, the device that provides the SAP 204 to the fibrous matrix 202 may be a operated at a relatively continuous, stable rate to provide consistency even at relatively high line speeds, and does not require the use of relatively imprecise or difficult to control devices that attempt to modulate the flow rate or positioning of the SAP 204 in the fibrous matrix 202. In addition, the use of folds 210 provides greater access to the SAP 204 and may reduce gel blocking, as described elsewhere herein. The present invention also may provide numerous other benefits with respect to manufacturing and garment performance and in other regards.

Other embodiments, uses and advantages of the invention will be apparent to those skilled in the art from consideration of the specification and practice of the invention disclosed herein. The specification should be considered exemplary only, and the scope of the invention is accordingly intended to be limited only by the following claims.

I claim:

1. An absorbent core having a longitudinal dimension and a lateral dimension, the absorbent core comprising:
a fibrous matrix;
superabsorbent particles distributed within the fibrous matrix; and
a folded zone having at least one laterally-extending fold;
wherein the folded zone extends through at least a portion of the longitudinal dimension of the absorbent core.

2. The absorbent garment of claim 1, wherein the folded zone extends through substantially the entire lateral dimension of the absorbent core.

3. The absorbent core of claim 1, further comprising:
a first tissue layer disposed on a first side of the fibrous matrix; and
a second tissue layer disposed on a second side of the fibrous matrix.

4. The absorbent core of claim 3, wherein the first tissue layer and the second tissue layer are portions of a single tissue sheet.

5. The absorbent core of claim 1, wherein the folded zone comprises two or more laterally-extending folds.

6. The absorbent core of claim 5, further comprising a laterally-extending void between each adjacent pair of laterally extending folds.

7. The absorbent core of claim 1, wherein each laterally-extending fold has a length of about 10 mm to about 300 mm.

8. The absorbent core of claim 1, wherein the folded zone comprises about 5 to about 15 laterally-extending folds, each laterally-extending fold having a length of about 20 mm to about 50 mm.

9. The absorbent core of claim 1, wherein the fibrous matrix comprises tow fibers.

10. The absorbent core of claim 9, wherein the tow fibers comprise cellulose acetate.

11. The absorbent core of claim 1, wherein the superabsorbent particles and fibrous matrix are a uniform basis weight fibrous matrix/superabsorbent polymer mixture.

12. The absorbent core of claim 1, wherein the folded zone has greater absorbency than other portions of the absorbent core.

13. An absorbent garment comprising:
a topsheet;
a backsheet; and
an absorbent core having a longitudinal dimension and a lateral dimension, the absorbent core being disposed between the topsheet and the backsheet and comprising a fibrous matrix and superabsorbent particles distributed within the fibrous matrix;
wherein the absorbent core comprises a folded zone comprising at least one laterally-extending fold, the folded zone extending through at least a portion of the longitudinal dimension of the absorbent core.

14. The absorbent garment of claim 13, wherein the folded zone extends through substantially the entire lateral dimension of the absorbent core.

15. The absorbent garment of claim 13, wherein the absorbent core further comprises:
a first tissue layer disposed on a first side of the fibrous matrix; and
a second tissue layer disposed on a second side of the fibrous matrix.

16. The absorbent garment of claim 13, wherein the first tissue layer and the second tissue layer are portions of a single tissue sheet.

17. The absorbent garment of claim 13, wherein the folded zone comprises two or more laterally-extending folds.

18. The absorbent garment of claim 17, further comprising a laterally-extending void between each adjacent pair of laterally extending folds.

19. The absorbent garment of claim 13, wherein each laterally-extending fold has a length of about 10 mm to about 300 mm.

20. The absorbent garment of claim 13, wherein the folded zone comprises about 5 to about 15 laterally-extending folds, each laterally-extending fold having a length of about 20 mm to about 50 mm.

21. The absorbent garment of claim 13, wherein the fibrous matrix comprises tow fibers.

22. The absorbent garment of claim 21, wherein the tow fibers comprise cellulose acetate.

23. The absorbent garment of claim 13, further comprising an insult point on the topsheet disposed proximal to where exudates from an intended wearer initially strike the topsheet when the garment is worn by the intended wearer, and wherein the folded zone is disposed adjacent to the insult point.

24. The absorbent garment of claim 13 wherein the superabsorbent particles and fibrous matrix are a uniform basis weight fibrous matrix/superabsorbent polymer mixture.

25. The absorbent garment of claim 13, wherein the folded zone has greater absorbency than other portions of the absorbent core.

26. An apparatus for providing folded zones in a continuous core supply moving from an upstream location to a downstream location, the apparatus comprising:
a folding means adapted to provide one or more creases in the continuous core supply;
a flattening means, positioned downstream of the folding means, to flatten the one or more creases to be substantially parallel with the continuous core supply; and
a tensioning means, positioned upstream of the folding means, to maintain tension in the continuous core supply.

27. The apparatus of claim 26, wherein the folding means comprises a pair of counter-rotatable folding rolls, each folding roll comprising one or more teeth, the teeth of each counter-rotatable folding roll being positioned to mesh the teeth of the other counter-rotatable folding roll.

28. The apparatus of claim 26, wherein the flattening means comprises a pair of press rolls.

29. The apparatus of claim 26, wherein the tensioning means comprises a tension arm.
